(19) United States
(12) Patent Application Publication
(10) Pub. No.: US 2010/0004656 A1
(43) Pub. Date: Jan. 7, 2010

(54) BONE CEMENT CONDUCTOR DEVICE

(75) Inventor: Geraldo MARINS DOS REIS, JR., Campinas (BR)

Correspondence Address: OBLON, SPIVAK, MCCLELLAND MAIER & NEUSTADT, L.L.P., 1940 DUKE STREET, ALEXANDRIA, VA, 22314, US

(73) Assignee: GM DOS REIS JUNIOR, Campinas (BR)

(21) Appl. No.: 12/498,044

(22) Filed: Jul. 6, 2009

(30) Foreign Application Priority Data
Jul. 4, 2008 (BR) PI 0802530-4

(51) Int. Cl. A61B 17/58

(52) U.S. Cl. 606/93

(57) ABSTRACT

A bone cement conductor device provides for the application and the safe and effective filling of bone cement during surgical treatment procedures in patients suffering from osteoporosis. More specifically, the device is constituted by a structure that is easy to manufacture and handle, and that facilitates surgical processes and treatments, demanding less effort of surgeons. The device includes an outer encasement with a conductor body being provided with interlink and connection mechanisms with a cement injection device and a bone screw that eliminate any risk of leakage or incorrect application of the cement in the patient's bones.

### FIELD OF THE INVENTION

The present invention refers to a cement conductor device for processes of attaching osseous synthesis in patients whose bone structure is compromised and lacks the sufficient resistance for attaching bone screws, such as patients suffering from osteoporosis.

More particularly, the present invention refers to a guide that interlinks the cement injection device to the bone screws, said conductor device having structural characteristics that ensure the application of cement in the damaged bone structures by way of the body of the bone screw, which is fixed adequately in the desired position and, also, safely and effectively.

Additionally, the cement conductor device, that is the object of the present invention, comprises connection mechanisms which conduct the cement between the applicator device and the bone screw efficiently and safely, eliminating the risks of leakage of the material during application.

### BACKGROUND OF THE INVENTION

As known to persons skilled in the art, osteoporosis is a disease that affects the bones and is caused by natural aging of the human being, and is characterized by the reduction in bone mass that generates hollow, fragile and fracture-prone bones. Currently, there is a series of treatments and diagnoses for early identification of osteoporosis, through medicines and surgical interventions.

However, the problem of patients suffering from this kind of disease is worsened when the need arises for the introduction and attachment of osseous synthesis of the bone plate kind, intervertebral spacers and implants, among others, which are fixed to the bone structures by means of bone screws.

In such situations, it is noted that the bone screws, when introduced into the bone structure of the patient with osteoporosis, are not fixed safely. Normally, the bone screws end up loose, lax, damaging the bone structure, since these bones are relatively hollow, that is, there is no bone matter to anchor the bone screws. As a result, it is not possible to establish a firm, safe and stable attachment of the bone screw.

In order to solve the problem of attaching and anchoring the bone screws on these bone structures compromised by osteoporosis, during surgical treatments, doctors introduce the matter called “bone cement” or “surgical cement”. The purpose of this material is to rebuild the structure of the bone at the site where the bone screws are fixed and anchored.

One of the ways of proceeding with the introduction of this bone cement is through an opening in the bone and, sequentially, said cement is introduced to rebuild and fill in the compromised bone structure. Before said cement hardens, the doctors need to introduce the bone screw in such a way that it remains duly positioned in the bone.

However, as persons skilled in the art will appreciate, this kind of procedure may lead to certain drawbacks for doctors and chiefly for patients, since: i) the cement may harden before the due positioning of the bone screw; ii) it requires a relatively large incision in the patient; iii) it increases the risk of infection due to the size of the incisions; iv) it requires extra attention in rebuilding the bone structure with cement; v) there is a risk that the bone cement may accidentally be applied in an inappropriate place, which may compromise the patient's health.

As can be noted, this technique of applying and manual filling of the bone structures with cement generates a series of problems for patients, because it increases the surgery time, the risk of infection due to the size and prolonged exposure time of the incision, the post-operation recovery time and, principally, it may lead to inadequate positioning of the bone screws.

In order to solve some of the drawbacks referred to above, today cement applicator devices are used. They are encased directly in bone screws, making the application of the cement be carried out by means of the very body of the bone screw. Said bone screw has an inside channel which interlinks a series of exit holes disposed in the threaded portion of the bone screw.

Although it solves the problem of hardening of the cement before introducing and positioning the bone screw, since the cement is only introduced after the due positioning of the bone screw in the bone structure, it can be verified that in practice such disposition is neither efficient nor easy to apply.

More specifically, the difficulties observed in practice are related to incompatibility of connections between the cement injection device and the bone screw, but principally in relation to the efficiency and safety during the process of applying the cement in the bone structure.

As persons skilled in the art will appreciate, the cement injection device has similar characteristics to that of a syringe constituted by a cylindrical body that internally accommodates a mobile piston and comprises an end with an opening to extract the material. This end is coupled directly onto the interface connector of the bone screw, or on the screw head itself, without any safety mechanism for conducting the cement that leaves the injection device towards the bone screw channel.

Additionally, since there are no appropriate devices for conducting and adequately applying cement, it can be seen that doctors face a major hurdle in obtaining a precise alignment between the opening of the material extraction of the injection device and the bone screw channel.

Accordingly, it can be verified that all devices and procedures of applying bone cement or surgical cement known in the state of the art reveal problems relating to risks of causing leakage during surgical procedures, allowing the material to be applied at inappropriate sites inside the incisions.

In view of the foregoing, it is clear that there is no bone cement conducting devices which reveal a simple structure and, principally, efficient to the point of ensuring due application of cement in the bones, as well as safe anchoring of the screw in the weakened bone.

### BRIEF DESCRIPTION OF THE INVENTION

Therefore, in order to overcome the deficiencies and solve the drawbacks mentioned above, identified in the bone cement applicator mechanisms in the state of the art, the bone cement conductor device that is the object of the present invention was developed.

More specifically, it is an objective of the present invention to provide a bone or surgical cement conductor device, that allows the application of this material in the bone structure during surgical processes adequately and safely, since it comprises an arrangement capable of promoting an efficient and safer connection between the cement applicator device and the bone screw.

Further, another objective of the present invention is a cement conductor device that presents a simple structure that is easy to manufacture and to assemble.

Another objective of the present invention is to provide a bone cement conductor device comprising an arrangement that is easy for surgeons to handle during surgical processes, eliminating any risk of leakage and, ensuring that the material applied be introduced only in the desired place.

To achieve the objectives proposed above, the bone cement conductor device, according to the present invention, comprises a structure made up of an outer encasement that involves a conductor body, also being constituted of safe and adequate connection and interlinking mechanisms between the bone screw and the material outlet end of the injection device per se.

### BRIEF DESCRIPTION OF THE DRAWINGS

The objectives, improvements and technical effects provided by the bone cement conductor device, that is the object of the present invention, will be apparent to persons skilled in the art from the detailed description below making references to the schematic drawings appended hereto, wherein:

FIG. 1 depicts a perspective view of the bone cement conductor device of the present invention connected between the injection device and the bone screw;

FIG. 2 depicts a perspective view of the bone cement conductor device according to the present invention;

FIG. 3 depicts another perspective view of the device illustrated in FIG. 1;

FIG. 4 shows an exploded side view of a preferred embodiment of the bone cement conductor device according to the present invention;

FIG. 5 depicts an exploded view of the bone cement conductor device.

### DETAILED DESCRIPTION OF THE INVENTION

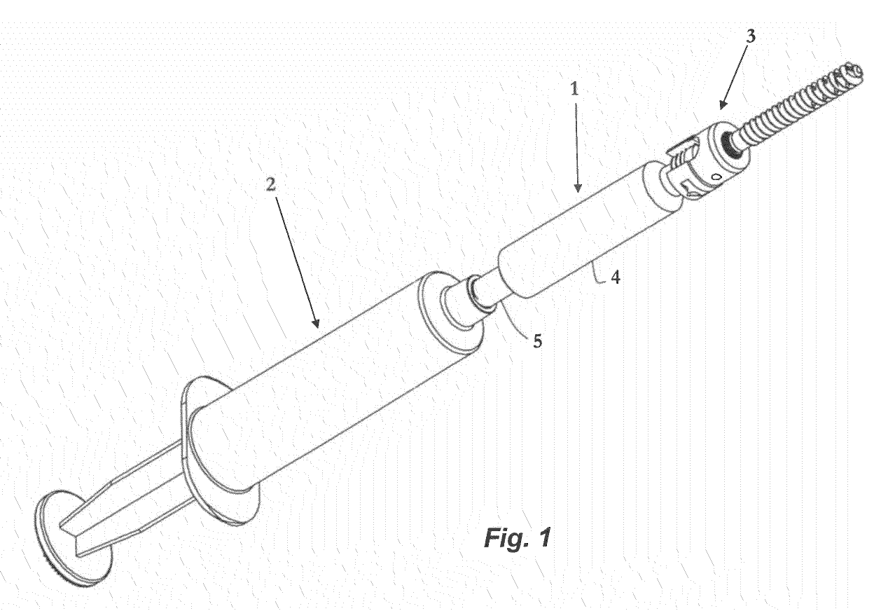

According to FIG. 1, it is noted that the bone cement conductor device (1) that is the object of the present invention, is an element that connects the injection device (2) and the bone screw connector (3). More particularly, the conductor device (1) comprises an outer encasement (4) that involves a conductor body (5) whose ends (6, 7) are exposed for due encasement with the injection device (2) and the bone screw (3).

As persons skilled in the art will appreciate, the injection devices (2) and the bone screws (3) are known surgical instruments that have been used for years and, for this reason, it is understood that there is no need to provide more specific details thereon. It is important to point out that the injection devices (2) are provided with an applicator nozzle provided with connection means of the thread type, and the bone screws (3) have an end, called interface connector, which can be the very head of the screw itself. These connectors comprise a cylindrical format with semi-open side walls and internally have means for attaching the osseous synthesis, for example a threaded surface or any suitable means of adaptation commonly used in surgical treatments.

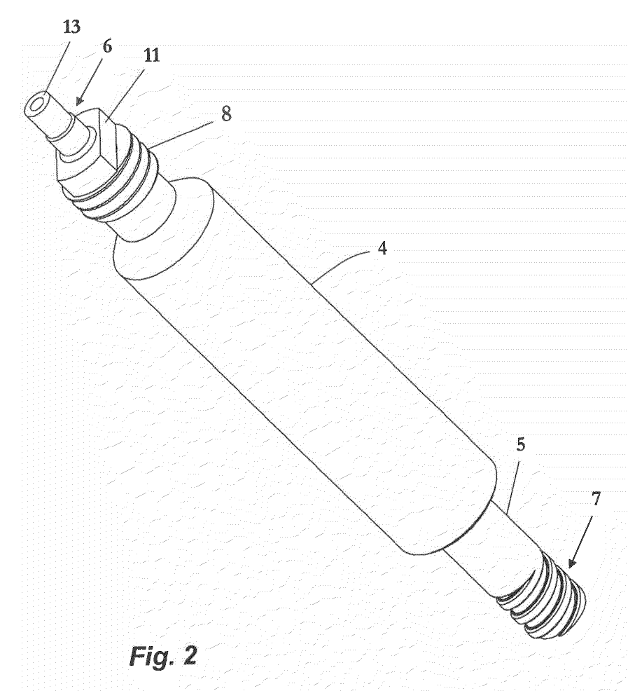

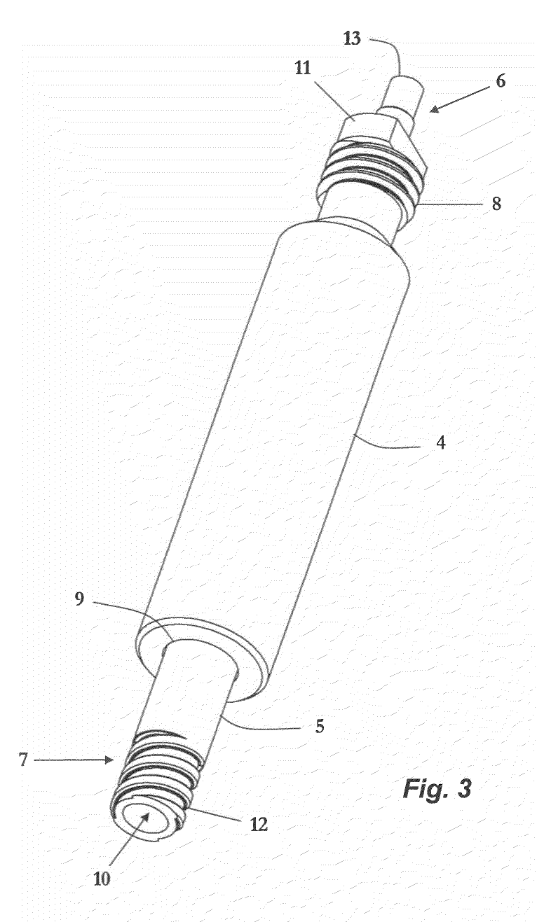

More particularly in relation to FIGS. 2 and 3, it is observed that the outer encasement (4) comprises, preferably, a cylindrical format in which one of its ends has a connection mechanism (8) whose purpose is to promote the encasement and attachment of the conductor device (1) in the interface connector or in the bone screw head. Commonly, the connector devices of the bone screws (3) are provided with inside threads, which is why the connection mechanism (8) of the outer encasement (4) should correspond to these characteristics, that is, a thread corresponding to the inside thread of the connector device of the bone screw (3).

Nevertheless, it must be made clear that although the drawings depict threads as connection mechanisms, any other form of connecting the connection mechanism (8) of the outer encasement (4) onto the bone screw (3) can be used, provided that there is due correspondence between the connections.

At the opposite end to the connection mechanism (8) the outer encasement (4) has an opening (9) for the passage of the conductor body (5), as depicted in FIG. 3.

The conductor body (5), preferably, comprises a cylindrical format and has an inside channel (10) for the passage of cement. The ends (6, 7) that remain exposed outside of the encasement (4) are respectively provided with locking and anchoring mechanism (11) and adaptation mechanism (12) with the outlet nozzle of the injection device (2) material. Additionally, at the end (6) of the conductor body (5) is disposed a cement outlet nozzle (13) which, during surgical treatments, fits into the bone screw head.

Said locking and anchoring mechanism (11) comprises a format that encases in the openings formed in the connectors of the bone screws (3), to enable the aligning between the outlet nozzle (13) and the cavity of the bone screw head (3). Preferably, but not limitedly in the present invention, said locking and anchoring mechanism (11) is shaped like a parallelepipedon to fit precisely into the open walls of the connector of the bone screw (3).

However, as persons skilled in the art are aware, there are various models and formats of bone screws used to attach osseous synthesis, each comprising specific formats for the application thereof. Thus, it is clear that the format of the locking and anchoring mechanism (11) may comprise any format provided that it is compatible with the format of the screw head and/or of the connector of the screw (3), for example: triangle, star, square, circle, fissure, cross, torque, hexagon etc.

The purpose of the adaptation mechanism (12) disposed at the end (7) of the conductor body (5) is to encase the conductor device of the present invention at the outlet end of the injection device (2). Normally this outlet end of the injection device (2) has an ejector nozzle enveloped by a cylindrical wall having an inside thread, in this case, for example, the adaptation mechanism (12) should be a corresponding thread.

At this stage, it is also important to clarify that other means of adaptation can be implanted in the present invention, for example, the encasement combination of the click fit or snap fit kind or any other equivalent means, only limited by the fact that there must be compatibility between the connections of the conductor device (1) and the injection device (2).

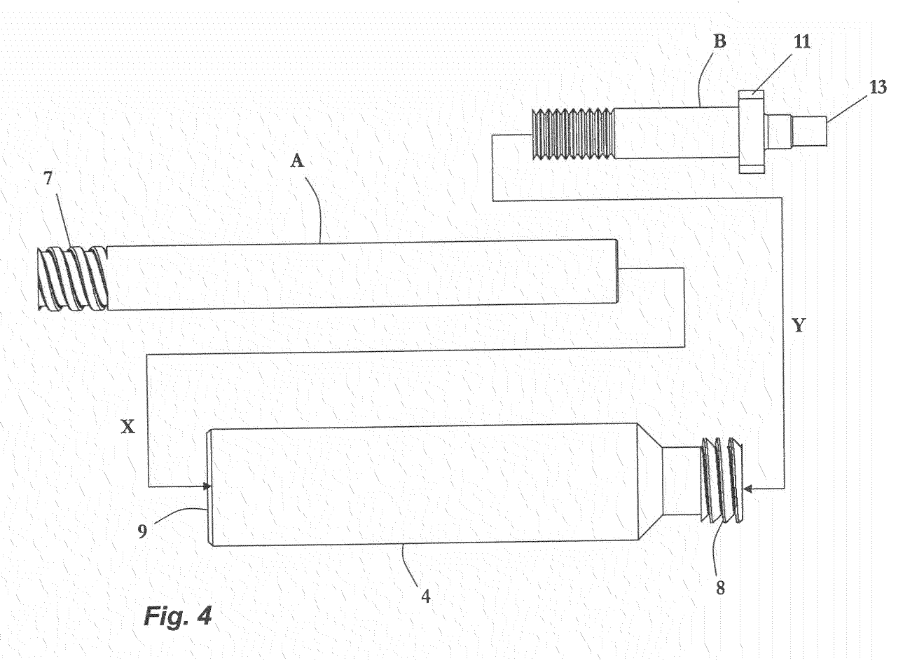

FIG. 4 shows an advantageous embodiment for the manufacture and assembly of the bone cement conductor device that is the object of the present invention. More particularly, in this embodiment of the conductor device (1) is constituted of three parts, namely: outer encasement (4), rear portion (A) and front portion (B) of the conductor body (5), the two latter being interlinked and fixed together, when disposed inside the encasement (4).

According to the indicative arrows (X, Y) it can be noted that the rear portion (A) is introduced into the outer encasement (4) through the opening (9), and the front portion (B) is introduced through the end of the encasement (4) having the connection mechanism (8). Internally, both portions (A, B) are connected and fixed together, forming the conductor body (5). Preferably, said portions (A, B) of the conductor body (5) are fixed together by means of threads, glue, encasement, solder or, any means that ensures the connection thereof.

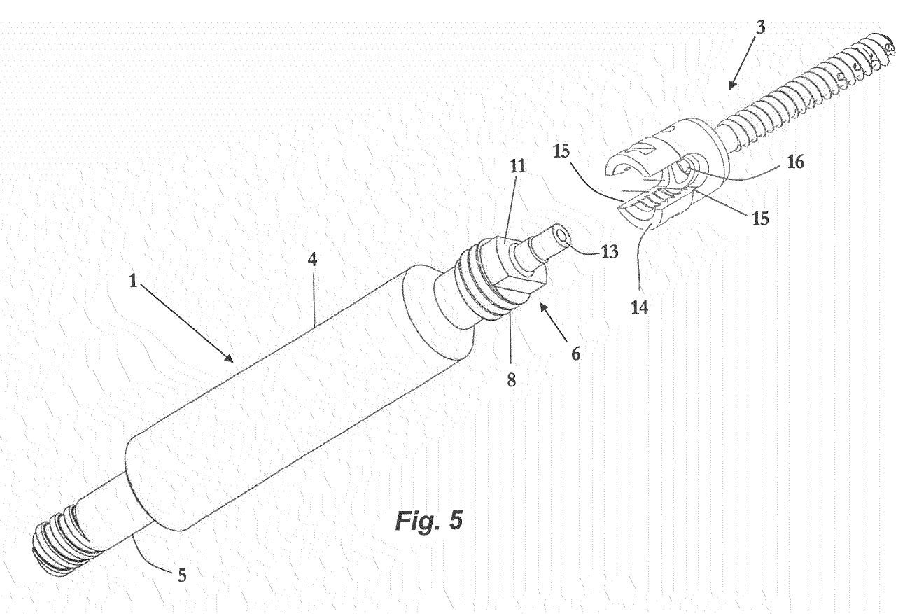

In reference to FIG. 5, next is a description of how to assemble the conductor device (1) onto the bone screw (3). More specifically:
the end (6) of the conductor body (5) is coupled to the connector (14) of the bone screw (3);
the locking and anchoring mechanism (11) is aligned on the openings (15) of the connector (14) of the bone screw, blocking the movement of the conductor body (5) in relation to the bone screw (3); and
the encasement (4) is rotated, meaning the connection mechanism (8) encases in the corresponding connection of the connector of the bone screw and the outlet nozzle (13) is duly accommodated in the cavity (16) of the bone screw head.

At the opposite end of the cement conductor device, according to the present invention, the injection device (2) can be coupled as indicated previously, by means of threads, encasement etc.

Thus, after assembling the due connections of the cement conductor device of the present invention, it is possible to obtain a bone cement applicator capable of conducting and applying the cement safely and effectively, without causing any risk of undesirable leakage during surgical treatments.

Additionally, it must be emphasized that the embodiment indicated in FIG. 4 is merely a preferred form of construction, since it is possible to obtain a conductor body in a single part or, also with more than two parts that will be grouped during the manufacturing and assembly process of the device.

Although the embodiments identified in the appended drawings depict a cylindrical-shaped encasement, other forms could be used, in order to obtain a simpler format to handle the device or else to obtain a different aesthetic characteristic, since said encasement just needs to be able to envelop and be free to turn around the conductor body.

It is also worth while mentioning that preferably, the conductor device that is the object of the present invention is manufactured entirely of plastic, but obviously other materials could be used, such as metal, or a combination of these materials.

Additionally, the dimensions of the device may undergo alterations to meet the needs and suit the interests of doctors and also of the manufacturers themselves.

Therefore, by virtue of the foregoing, it is clear that the bone cement conductor device, according to the present invention, comprises characteristics capable of providing various advantages and technical/functional effects that increase the safety, efficiency and agility in the surgical processes and treatments that require the application and filling of bone cement in patients suffering from osteoporosis. Besides comprising a structure that is easy to manufacture and handle.

Lastly, it ought to be made clear that the terms bone or surgical cement is a material, the purpose of which is to promote the filling of the bone structure and, preferably, in surgical cases where the patient suffers from osteoporosis. Therefore, as persons skilled in the art likely know, there is a plurality of compatible materials and currently used for this purpose, which should be considered here as being within the definition of said term bone or surgical cement.

As persons skilled in the art will appreciate, there are countless possible modifications and variations of the present invention in light of the teachings above, without departing from the scope of protection, as delimited by the claims appended hereto.

1. A bone cement conductor device, comprising:
an outer encasement including a conductor body provided with two ends which are exposed outside of said encasement, a locking and anchoring mechanism, an adaptation mechanism, a connection mechanism at one of the ends, and an opening at an other end for a passage of said conductor body.

2. The bone cement conductor device according to claim 1, wherein said outer encasement is cylindrical.

3. The bone cement conductor device according to claim 1, wherein said conductor body is formed of a single part.

4. The bone cement conductor device according to claim 1, wherein said conductor body is formed of two or more parts.

5. The bone cement conductor device according to claim 4, wherein the conductor body includes a rear portion and a front portion.

6. The bone cement conductor device according to claim 5, wherein said front and rear portions are connected and fixed together, inside the outer encasement.

7. The bone cement conductor device according to claim 5, wherein said front and rear portions are connected and fixed together inside the outer encasement by threads, encasement, glue, solder, or a combination thereof.

8. The bone cement conductor device according to claim 1, wherein one of the ends of the conductor body includes a cement outlet nozzle configured to fit into a hole of a bone screw head.

9. The bone cement conductor device according to claim 8, wherein said locking and anchoring mechanism is compatible with a connector of the bone screw head.

10. The bone cement conductor device according to claim 9, wherein said locking and anchoring mechanism is configured to fit into openings formed in connectors of bone screws.

11. The bone cement conductor device according to claim 1, wherein said locking and anchoring mechanism has a parallelepipedon, triangle, star, square, circle, fissure, cross, toque or hexagon shape.

12. The bone cement conductor device according to claim 1, wherein the adaptation mechanism is disposed at one of the ends of the conductor body and provides compatibility between connections of the conductor device and an injection device.

13. The bone cement conductor device according to claim 1, wherein the conductor device is manufactured entirely of plastic, metal, or a combination thereof.
